(19) United States
(12) Patent Application Publication
(10) Pub. No.: US 2023/0127231 A1
(43) Pub. Date: Apr. 27, 2023

(54) PROCESSING UNIT, SYSTEM, AND COMPUTER-IMPLEMENTED METHOD FOR A VEHICLE INTERIOR FOR DETECTING AND REACTING TO ODORS OF A VEHICLE OCCUPANT

(71) Applicant: ZF Friedrichshafen AG, Friedrichshafen (DE)

(72) Inventor: Rene SEIDEMANN, Friedrichshafen (DE)

(73) Assignee: ZF Friedrichshafen AG, Friedrichshafen (DE)

(21) Appl. No.: 17/913,121

(22) PCT Filed: Mar. 10, 2021
(86) PCT No.: PCT/EP2021/055990

§ 371 Date: Sep. 20, 2022

(30) Foreign Application Priority Data
Mar. 20, 2020 (DE) 10 2020 203 584.9

(51) Int. Cl. B60R 25/25; G01S 17/88; B60R 25/04; B60R 25/30; G06V 20/70; G06V 20/59; G06V 40/16; G06V 10/26; A61B 5/00

(52) U.S. Cl. CPC B60R 25/25; G01S 17/88; B60R 25/04; B60R 25/305; G06V 20/70; G06V 20/59; G06V 40/172; G06V 10/26; A61B 5/4011; A61B 5/6893

(57) ABSTRACT

The invention relates to a processing unit, a system, and a computer-implemented method for a vehicle interior, for detecting and reacting to odors of a vehicle occupant.

The invention relates to a processing unit, system, and computer-implemented method for a vehicle interior, for detecting and reacting to odors of a vehicle occupant.

Odor sensors are known from the prior art, e.g. U.S. 2019/0227042 A1. The known odor sensors function on the basis of light absorption spectroscopy, surface adsorption methods or chemical reactions.

Vehicle alcolocks, also referred to as breath alcohol ignition interlock devices, are known from the prior art, which lock the ignition in a vehicle if the driver's alcohol content exceeds a specific value, e.g. from press releases by the European Parliament. The known alcolocks comprise a handheld device accessible from the driver's seat that measures the alcohol concentration in an exhalation. There are also devices placed in the region of the dashboard or steering wheel that measure exhalations. These types of hand-held devices and installations are uncomfortable to use.

The object of the invention is to better detect odors from vehicle occupants, in particular the driver, such that it is possible to more effectively immobilize the vehicle.

According to one aspect of the invention, there is a processing unit for a vehicle interior for detecting and reacting to odors of a vehicle occupant. The processing unit comprises a first interface to a first sensor, which records odor molecules in the exhalations and/or body odors of the vehicle occupants in a vehicle interior and converts these into first signals in order to obtain initial signals. The first signals are the results of the effects of light interactions or comprise frequencies and/or amplitudes of oscillations. The processing unit also comprises a second interface to a second sensor, which identifies the vehicle occupants, in order to obtain identities of the vehicle occupants. The processing unit executes commands with which the processing unit assigns odors indicating drugs and/or illnesses to the identities of the vehicle occupants on the basis of a position of the vehicle occupant in the vehicle interior, and generates a second signal in the case of a positive assignment, in which the commands comprise a first machine learning algorithm that is trained to identify alcohol, cocaine, amphetamines, cigarette smoke, cannabis, tetrahydrocannabinol, morphine, methadone, ammonia, acetone, or a combination of these substances in the odors of the vehicle occupants, on the basis of the first signals. The processing unit also comprises a third interface to at least one vehicle unit for providing the second signal to the vehicle occupants and/or a vehicle control system. The first machine-learning algorithm is trained to identify the vehicle occupants on the basis of semantic image segmentation. Alternatively, the commands comprise a second machine-learning algorithm that is trained to identify vehicle occupants on the basis of semantic image segmentation.

The processing unit is a computer unit that processes input signals and issues output signals, e.g. in the form of regulating and/or control signals.

According to one aspect of the invention, the processing unit comprises an electronic circuit, e.g. an integrated circuit. By way of example, the processing unit comprises programmable logic modules, e.g. field programmable gateway arrays, in which circuit structures can be programmed using hardware commands. According to one aspect of the invention, the processing unit comprises at least one central processing unit that executes software program commands, and/or at least one graphics processing unit for executing processes in parallel, or at least one multi-core processor. According to another aspect of the invention, the processing unit comprises at least one memory, e.g. a RAM, DRAM, SDRAM, or SRAM, for signal and/or data exchange with processors. According to one aspect of the invention, the processing unit is a system on a chip. This means that all, or at least most, of the functions are integrated on one chip, and can be expanded modularly. According to another aspect of the invention, the processing unit or the chip is integrated in an electronic control unit, e.g. an electronic control unit for vehicle control.

According to one aspect of the invention, the processing unit implements an artificial neural network, which simulates mitral cells, apical and lateral dendrites of the mitral cells, the respective soma of the mitral cells, and granule cells of the olfactory bulb. This means that the artificial neural network is programmed, or executed on the processing unit, such that it simulates the mammalian olfactory system. By way of example, the odor molecules from the exhalations are provided to the apical dendrites of each mitral cell. The mitral cells are neurons in the artificial neural network. The apical dendrites correspond to weighted connections between mitral cell neurons. The mitral cell neurons are activated through activation functions in the artificial neural network, for example, and activate other neurons in another layer of the artificial neural network. The other neurons in the other layer correspond to the granule cells of the olfactory bulb.

According to another aspect of the invention, the processing unit comprises a neural circuit that simulates a mammalian olfactory system. According to one aspect of the invention, the neural circuit is a neuromorphic circuit. The mammalian olfactory system is reproduced by the neuromorphic circuit in the form of hardware. The neuromorphic circuit is formed using CMOS technology, for example, i.e. it is based on complementary metal-oxide-semiconductors. The neuromorphic circuit comprises neuristors, i.e. components comprising neuron models and synapses. The elements of the neuromorphic circuit simulate, e.g., mitral cells, apical and lateral dendrites of the mitral cells, the respective soma of the mitral cells, and granule cells of the olfactory bulb. According to another aspect of the invention, the elements of the neuromorphic circuit are connected in an artificial neural network. According to another aspect of the invention, the artificial neural network, which simulates the mammalian olfactory system, is implemented on the neuromorphic circuit.

According to one aspect of the invention, the processing unit implements an artificial neural network that simulates the mitral cells, apical and lateral dendrites of the mitral cells, the respective soma of the mitral cells and granule cells of the olfactory bulb. This means that the artificial neural network is programmed or executed on the neuromorphic circuit such that it simulates the mammalian olfactory system. By way of example, the odor molecules from the exhalations are provided to the apical dendrites of each mitral cell. The mitral cells are neurons in the artificial neural network. The apical dendrites correspond to weighted connections between mitral cell neurons. The mitral cell neurons are activated through activation functions in the artificial neural network, for example, and activate other neurons in another layer of the artificial neural network. The other neurons in the other layer correspond to the granule cells of the olfactory bulb.

Vehicles comprise passenger vehicles, e.g. passenger automobiles, minibuses, busses, and light and heavy-duty utility vehicles, farm machines, e.g. tractors or excavators, rail vehicles, e.g. trains, ships, and passenger drones. The invention is therefore not limited to passenger automobiles, but instead can be advantageously used with many types of vehicles that are driven by a person with respect to detecting and reacting to odors. According to another aspect, the invention can be advantageously used by fleet operators with respect to detecting and reacting to odors. By way of example, the vehicles owned by a fleet operator can be equipped with the subject matter of the invention. This allows the fleet operators to obtain information regarding their drivers pertaining to driving fitness on the basis of alcohol consumption or illnesses detected by the subject matter of the invention. According to one aspect of the invention, the detections according to the invention are sent to the fleet operator, e.g. using electronic logbook entries and/or via wireless communication.

Odors comprise breath odors and odors from other bodily orifices in living beings, i.e. body odors, e.g. perspiration. If there are any animals in the vehicle, e.g. dogs, the first sensor will also detect animal odors. The functionalities of the first sensors do not allow for a distinction to be made regarding the source of an odor, e.g. human or animal. The second sensors distinguish between the different beings. This means that the combination of the functionalities of the first sensors and second sensors allows for the odors to be assigned to the respective living beings.

Vehicle occupants comprise vehicle drivers, vehicle occupants not driving the vehicle, e.g. passengers in the front and back seats, and animals. The first sensors detect odors from the vehicle driver, passengers, and animals in the vehicle interior. According to one aspect of the invention, the odors from the vehicle drivers are evaluated.

The effects of light interactions comprise light diffusion, backscatter, reflection, transmission, deflection, and refraction. According to one aspect of the invention, the first signals are the result of light diffusion.

Odor molecules are detected by means of the signatures of the odor molecules, for example. The signatures comprise absorption lines in an absorption spectrum, specific diffused light for the respective molecules, e.g. Raman scattering or Rayleigh scattering, specific oscillation patterns based on molecule masses, or curves in impedance spectroscopy. The oscillation patterns are recorded, for example, using acceleration sensors, e.g. micro-electro mechanical systems.

According to one aspect of the invention, the signatures for the respective molecules are specific diffused light, e.g. diffused light from backscatter.

An odor molecule is a molecule with a characteristic smell. By way of example, the odor molecule (R)-(+)-limonene is the main aroma compound in lemons.

The signals are electric signals, by way of example. The electric signals comprise oscillation frequencies.

The vehicle occupants are identified according to one aspect of the invention through facial recognition, e.g. three dimensional facial recognition, in which the second sensor is a 3D or 2D sensor, or comprises numerous 2D sensors. Three dimensional facial recognition comprises recognition, classification and/or localization of individual points on the face, e.g. cheekbones or eye spacings, according to one aspect of the invention. According to another aspect of the invention, the vehicle occupants are identified using skin texture analysis. By way of example, the face of a vehicle occupant is identified by analyzing the skin texture thereof. Skin texture analysis comprises determining a structure of lines and pores.

The commands executed by the processing unit are program instructions or hardware commands. The commands are in the form of software code or machine code.

Machine learning is a technology in which computers and other data processing devices learn to perform tasks from data instead of being programmed for these tasks. The machine learning algorithms teach how to identify odors and/or vehicle occupants from data.

One advantage of the identification of odors that indicate drugs and/or illnesses in the exhalations and/or body odors of the vehicle occupants using the first machine learning algorithm is that no chemical processes are used, in which the alcohol content in an exhalation into a breathalyzer is determined, which are a source of discomfort for the subject, and no surface contact methods, e.g. adsorption methods, or invasive methods such as blood-alcohol tests, are needed. Exhalations and other body odors are continuously available and evaluated by the processing unit.

By way of example, the first machine learning algorithm comprises graphing a neural network, in which a graph is input to the input layer, the nodes of which simulate atoms and the lines of which simulate connections between molecules. The odor molecules are thus simulated in graphs, and the odors are determined using a graphing neural network. The graphing neural network has learned, for example, to determine the odors associated with the odor molecules from the structures of the odor molecules. This means of determining an odor is referred to as a quantitative structure or relationship, see, e.g., B. Sanchez-Lengeling et al., “Machine Learning for Scent: Learning Generalizable Perceptual Representations of Small Molecules,” arXiv: 1910.10685v2 [stat.ML].

Drugs and/or illnesses have characteristic odors that can be detected in exhalations. Drugs that can be detected in exhalations comprise alcohol, cocaine, amphetamines, cigarette smoke, cannabis, tetrahydrocannabinol, morphine, and methadone. By way of example, the smell of ammonia is an indication of kidney failure. The smell of acetone indicates diabetes.

If alcohol is detected in an exhalation, and identified as coming from the position of the vehicle driver, the second signal comprises an electric signal for a display, e.g. in the form of an infotainment system or heads-up display, or an acoustic device, e.g. speakers in the vehicle interior, to inform the vehicle occupants visually or acoustically that the driver is unfit due to alcohol content. The second signal also comprises a regulating and/or control signal for the vehicle control system that locks the vehicle ignition in this case. In addition to the display and acoustic device, a control unit for vehicle communication is another example of a vehicle unit. Vehicle communication comprises vehicle-to-everything and vehicle-to-vehicle communication. The vehicle communication informs a fleet operator, for example, i.e. the business that owns the vehicle, or a government agency, that a vehicle driver in one of the vehicles in the fleet is unfit for driving due to alcohol consumption.

By assigning the odors on the basis of the positions of the vehicle occupants in the vehicle interior to identities of the vehicle occupants, the odors are linked to the vehicle occupants with regard to driving times according to an aspect of the vehicle driver. The processing unit creates a reconciliation or matching of the odor to the vehicle driver in this manner. Based on the positions of the vehicle occupants, it is also possible to determine whether the vehicle occupant to which the odor has been assigned is located in the driver's position, e.g. in the driver's seat, and within the range of the first and/or second sensor. This advantageously prevents or obviates deception or misuse by the vehicle occupants with regard to the detection and assignment of the odor to the vehicle occupants. By way of example, the driving ability of a driver is impaired by alcohol consumption. A passenger, however, may be fit to drive. If it is not possible to assign the odor to specific vehicle occupants on the basis of their positions in the vehicle interior, a passenger would be able to assume the role of the driver and start the vehicle. The processing unit would evaluate the odor of the passenger, detect no drugs, and provide a second signal for starting the vehicle to the vehicle control system via the third interface. The impaired vehicle driver would then drive the vehicle. By assigning the odor to the identities of the vehicle occupants based on their positions in the vehicle interior, the processing unit recognizes that the detected odor belongs to the passenger and not the vehicle driver. The second signal for starting the vehicle is first issued when the odors of the vehicle driver have been recorded, and no drugs have been detected. This increases traffic safety, in particular also for automated driving systems that rely on a vehicle driver as a fallback measure.

According to another aspect of the invention, a system is provided for the vehicle interior for detecting and reacting to odors of a vehicle occupant. The system comprises at least one first sensor, which detects odor molecules in the exhalations and/or body odors of the vehicle occupants in the vehicle interior, and converts these to first signals. The system also comprises at least one second sensor, which identifies the vehicle occupants. The system also comprises a processing unit according to the invention, which is connected for signal transfer to the first and second sensors via first and second interfaces. The system outputs a second signal from the processing unit to an optical, acoustic and/or tactile information unit, or a vehicle control unit, via a third interface.

Another aspect of the invention is a computer-implemented method for detecting and reacting to odors from vehicle occupants. The method comprises the following steps:
obtaining first signals that describe odor molecules,
obtaining identities of the vehicle occupants,
identifying odors indicating drugs and/or illnesses on the basis of the first signals, using a first machine learning algorithm that is trained to identify odors comprising the odors of alcohol, cocaine, amphetamines, cigarette smoke, cannabis, tetrahydrocannabinol, morphine, methadone, ammonia, acetone, or combinations of these substances on the basis of the first signals,
assigning the odors to the identities of the vehicle occupants on the basis of the positions of the vehicle occupants in the vehicle interior,
generating a second signal in the case of a positive assignment, and
providing the second signal to at least one vehicle unit,
   wherein a processing unit according to the invention or a system according to the invention is used to execute the method.

According to another aspect of the invention, there is a computer program for detecting and reacting to odors from vehicle occupants. The computer program comprises commands with which the processing unit according to the invention executes the method according to the invention when the computer program is run on the processing unit.

Further embodiments of the invention can be derived from the dependent claims, the drawings, and the descriptions of preferred exemplary embodiments.

The machine learning algorithm trained in semantic image segmentation first classifies the living being that has been detected, i.e. as a human or animal, e.g. a dog. The body of the being is then identified, and body parts are classified. This results in a body model. The classified body parts comprise the face and appendages, e.g. hands. Various features of the face or appendages, e.g. hands, are then determined.

Semantic image segmentation enables facial recognition, appendage, e.g. hand, recognition, and identification via facial recognition and/or appendage, e.g. hand, recognition. By way of example, the first or second machine learning algorithm breaks down a three dimensional image of a vehicle interior into segments and identifies a vehicle occupant's face and then designates this portion of the image as a face, and does the same for appendages, e.g. hands. Further elements in the face are broken down into segments and identified, e.g. as the mouth, eyes, nose, ears, forehead, chin, and/or facial muscles, and their arrangement among themselves is determined. With hands, the number of fingers, the lengths and/or widths of individual fingers, fingernail structure, surface area of the hand, blood vessel patterns on the palms, the back of the hand, or in the fingers, are determined. These are biometric features with which the vehicle occupants are identified.

According to one aspect of the invention, the machine learning algorithm comprises an artificial neural network that is trained in semantic image segmentation. The artificial neural network is a convolutional neural network, e.g. a VGG network, see K. Simonyan et al., “Very Deep Convolutional Networks For Large-Scale Image Recognition,” arXiv:1409.1556v6 [cs.CV]. The artificial neural network is trained using monitored training according to one aspect of the invention. This makes it easier to follow the learning process than with unmonitored training, which has advantages with regard to the validation and safety assurance of the processing unit.

According to another aspect of the invention, the machine learning algorithm comprises a random forest classifier that comprises uncorrelated decision trees, which grow according to a specific randomization during a learning process. Each tree in this forest can make a decision for a classification, and the classification with the highest frequency is then the final classification. The advantages with a random forest are that it can be trained relatively quickly due to the relatively brief training and/or construction times for individual decision trees, the evaluations can be done in parallel because of the numerous trees, and important classifications, e.g. for facial recognition or appendage, e.g. hand, recognition, are recognized.

According to another aspect of the invention, the machine learning algorithm comprises a support vector machine classifier which subdivides numerous objects into classifications such that the classification boundaries are surrounded by a relatively large empty region.

According to another aspect of the invention, the machine learning algorithm, or the artificial neural network on the computer according to the invention, is trained in semantic image segmentation. It is advantageous for the training when the computer has a microarchitecture for parallel execution of processes, in order to be able to train the artificial neural network efficiently, using a large amount of data. Graphics processors comprise this type of microarchitecture. The trained network is implemented on the computer according one aspect of the invention.

According to another aspect of the invention, the processing unit is integrated in a vehicle or in a vehicle electrical system. According to one aspect of the invention, the first and second sensors are integrated in the vehicle electrical system. This simplifies communication between the sensors, the processing unit, and the vehicle control system. The vehicle electrical system is a CAN bus, for example. At least part of the communication between the sensors, processing unit, and vehicle control system is wireless according to one aspect of the invention, e.g. through Bluetooth Low Energy, ANT or ANT+, or some other wireless standard, e.g. for the 868 MHz band, with which it has been established that relatively high energy densities can be transmitted. By way of example, communication between the sensors and between the sensors and the processing unit take place wirelessly. According to one aspect of the invention, the communication between the processing unit and the vehicle control system is hard-wired in order to increase the operating reliability and to provide better protection against cyber-attacks.

In one embodiment of the system, the first sensor is a vehicle lidar sensor. The vehicle lidar sensor is designed to detect odor molecules on the basis of diffused light returning from the vehicle interior.

A vehicle lidar sensor is a lidar sensor intended for use in a vehicle environment, e.g. the vehicle interior. By way of example, vehicle lidar sensors emit laser pulses in wavelengths in the infrared spectrum, which are not harmful to humans.

By way of example, the vehicle lidar sensor comprises a CCD or CMOS chip with integrated evaluation electronics for spectroscopy, e.g. Raman spectroscopy. By way of example, the light wavelengths of the returning light, which comprises elastic backscatter through the odor molecules, are determined from the signals for the pixels in the CCD or CMOS chips, on the basis of which the odor molecules are identified.

In another embodiment of the system, the vehicle lidar system is designed to emit numerous light pulses of different wavelengths, and to detect the odor molecules from the returning light for each of the wavelengths. The vehicle lidar system comprises control electronics for emitting different wavelengths according to one aspect of the invention.

By way of example, the vehicle lidar sensor is designed to emit two light pulses of different wavelengths. A first wavelength is selected according to one aspect of the invention such that it is absorbed by the substance, the concentration of which is to be determined. The second wavelength is selected such that it is not absorbed, or is absorbed as little as possible. A concentration profile of the substance is then calculated from the incremental comparison of the differences in the absorption of the backscatter signals in the first and second wavelengths. By way of example, this results in a determination of the concentration of alcohol molecules in the exhalations or body odors of the vehicle occupants.

In another embodiment of the system, the vehicle lidar system comprises Q-switching. This Q-switching results in shorter light pulses. This also results in high peak performance with less energy. The lower energy output ensures that the vehicle lidar sensor is not harmful to the vehicle driver. The high peak performance enables identification of the odor molecules. The Q-switching forms an electro-optic modulator.

According to another aspect of the invention, the vehicle lidar sensor is enclosed.

In another embodiment of the system, the second sensor is a 2D or 3D camera sensor, radar sensor, or lidar sensor. The system is designed to identify the vehicle occupants using facial recognition. The 3D camera sensor is a time-of-flight sensor, for example.

By way of example, a three dimensional geometry of the face is determined using a time-of-flight method, which forms the basis for the functioning of the second sensor.

According to another aspect of the invention, the three dimensional geometry of the face is determined using structured light. Structured light is obtained, for example, using a 3D camera sensor or lidar sensor.

In another embodiment of the system, the system comprises a sensor that unifies the functionalities of the first and second sensors in that the sensor is a time-of-flight sensor, in which the first signals are obtained from the diffused light returning from the vehicle interior, and the vehicle occupants are identified from the flight times for the light pulses reflected on the surface of a body, e.g. the face of a vehicle occupant. The sensor is a time-of-flight camera or vehicle lidar sensor, for example.

There are vehicle systems that comprise interior monitoring systems for monitoring the state of alertness of a vehicle driver and for monitoring safety systems such as seatbelts. These interior monitoring systems comprise a time-of-flight camera or a vehicle lidar sensor. The invention proposes that these systems be configured to detect odors that indicate drugs and/or illnesses in the exhalations and/or body perspiration of vehicle occupants using this system in order to potentially prevent the vehicle from being operated. This requires no additional systems for providing, e.g., an alcohol-sensitive ignition lock.

In another embodiment of the system, the second sensor detects individual appendages. The system is designed to identify the vehicle occupants on the basis of these appendages. By way of example, hands may be recorded by a camera. The system then distinguishes the hands of the driver from those of a passenger. This measure further prevents misuse. This is of particular advantage if the first sensor is an apparatus that measures alcohol content when a finger is placed on it by determining the blood alcohol concentration on the basis of reflections or other light interactions.

In another embodiment of the system, when it is used in the vehicle interior, the first sensor is placed on a vehicle steering wheel in the dashboard, and the second sensor, or the single unified sensor, is placed on the vehicle steering wheel, the dashboard, the windshield, or in the roof of the vehicle.

In another embodiment of the system, the second signal is a control signal for an immobilizer that prevents the vehicle from being operated, and the second signal is sent to the immobilizer via the third interface, or the second signal is a control signal that triggers a fail-safe or fail-operational state for the vehicle. If alcohol has been detected in the exhalations and/or body odors, or if a threshold value has been exceeded, e.g. a legal limit, the invention forms an alcohol-sensitive immobilizer or an improved alcolock. “Fail-safe” means that the vehicle cannot be started if odors from the vehicle driver have been identified that indicate drugs and/or illness. “Fail-operational” means that the vehicle remains operational and continues moving until reaching a safe stopping place if odors have been identified from the vehicle driver indicating drugs and/or illness while underway.

In another embodiment of the system, the system comprises a memory and/or communication means with which identified odors from drugs and/or illnesses are stored and/or a business to which the vehicle belongs, or a government agency, is provided with information regarding the identified odor. This allows the business or government agency to stop the vehicle in question remotely, or trigger a fail-operational state.

By this means, commercial vehicle drivers can also be monitored by the company they are working for or by government agencies with regard to driving fitness.

The invention shall be explained below in reference to exemplary embodiments shown in the drawings. Therein:

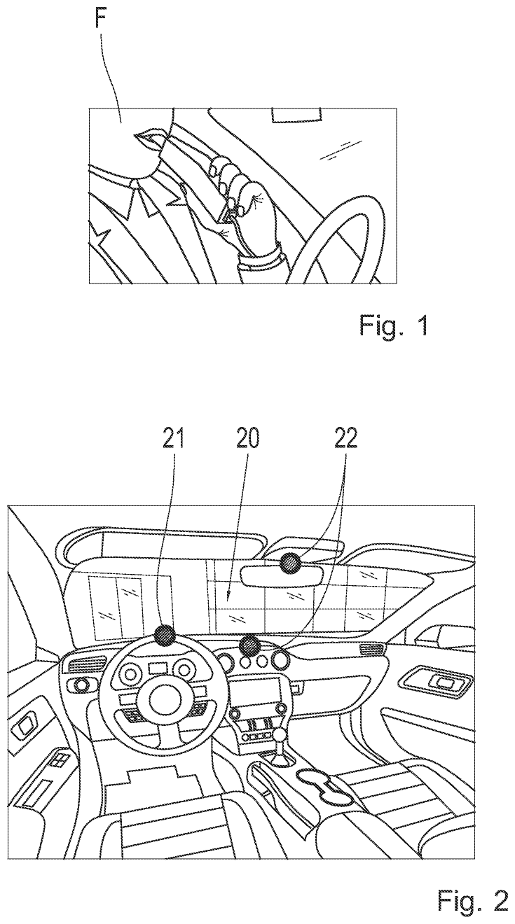

FIG. 1 shows an alcohol tester from the prior art;

FIG. 2 shows an exemplary embodiment of a system according to the invention in a vehicle interior;

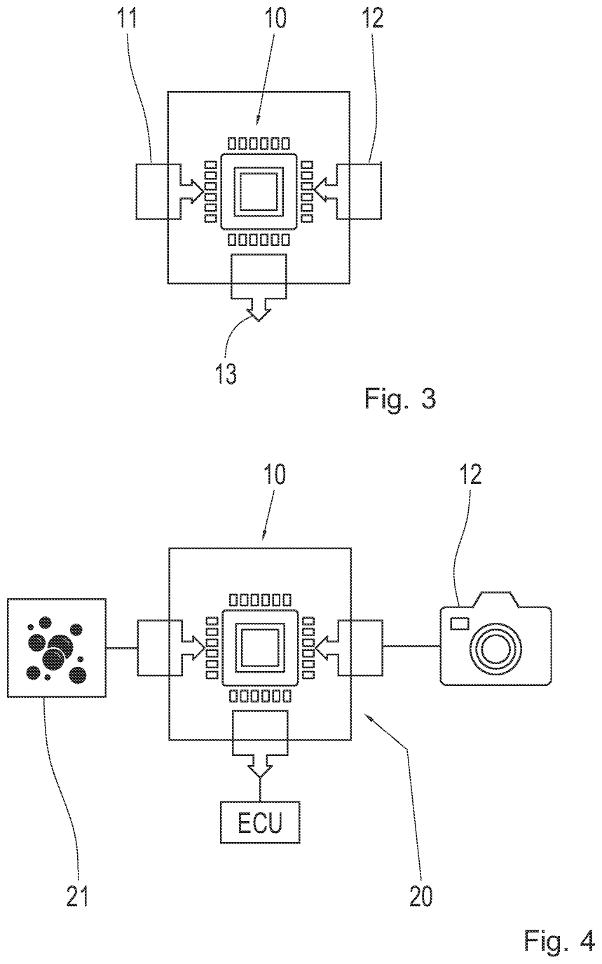

FIG. 3 shows an exemplary embodiment of a processing unit according to the invention;

FIG. 4 shows a schematic illustration of a system according to the invention;

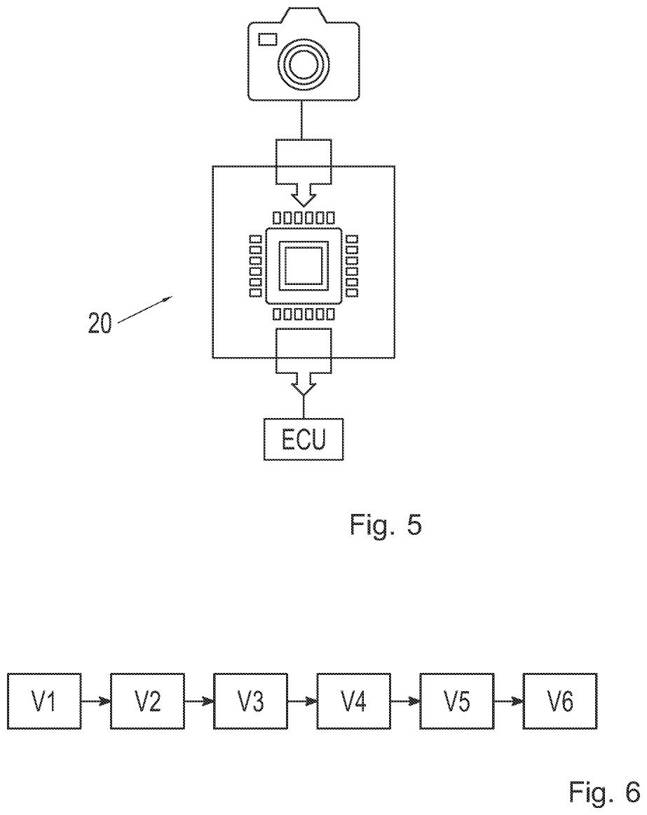

FIG. 5 shows a schematic illustration of another system according to the invention; and

FIG. 6 shows a schematic illustration of an exemplary embodiment of a method according to the invention.

The same reference symbols are used in the figures for the same or functionally similar elements. For purposes of clarity, only the relevant elements are indicated in the individual drawings.

FIG. 1 shows a vehicle driver as a vehicle occupant F, exhaling into a handheld device. The handheld device measures the alcohol concentration in the exhalation. If the value exceeds a threshold value, e.g. a legal limit, the vehicle starter motor will be locked. The vehicle will not be able to be started. Use of the handheld device is relatively uncomfortable. Moreover, the vehicle driver must make additional hand movements and/or manipulations in order to operate the handheld device before driving the vehicle.

The system 20 according to the invention is integrated in a vehicle interior in FIG. 2. FIG. 4 shows a schematic illustration of the system 20. The system 20 comprises a first sensor 21, by way of example. The first sensor 21 is integrated in the vehicle steering wheel and designed to detect odor molecules in the exhalations and/or body odor of the vehicle occupant F. The system 20 also comprises second sensors 22, which identify a vehicle driver's face using software for facial recognition. One of the second sensors 22 is located in the rear-view mirror in the vehicle interior and is a time-of-flight camera, for example. The other second sensor 22 is integrated in the dashboard and comprises, e.g., a camera, lidar, or radar sensor. The system 20 comprises a processing unit 10, as shown in FIG. 3.

The processing unit 10 receives signals from the first sensor 21 via a first interface 11. The processing unit 10 receives signals from the second sensor 22 via a second interface 12. Based on the signals, the processing unit detects odors in the exhalations and/or body odors of the vehicle occupants breathing toward the steering wheel, which indicate alcohol consumption, for example. The odors or the substances that cause the odors are identified using a machine learning method. The processing unit also links the odors to the identities of the vehicle occupants in order to prevent deception or misuse of the system 20. If it is determined, for example, that the alcohol concentration in the exhalations of the vehicle driver exceed a legal limit, the processing unit 10 generates a second signal. The second signal is sent to a vehicle control system ECU via a third interface 13 in the processing unit 10. The second signal causes the vehicle control system to prevent operation of the vehicle engine and/or drive train. The vehicle control system ECU is formed by an electronic control unit, for example.

FIG. 5 shows the system in an embodiment with just one sensor, e.g. a time-of-flight sensor or lidar sensor. In this case, the one sensor assumes the roles of both the first sensor 21 and the second sensor 22.

The method according to the invention is shown in FIG. 6. A first signal, describing odor molecules, is obtained in step V1. The vehicle occupants are identified in step V2. Odors indicating drugs and/or illnesses are identified in step V3 on the basis of the first signal using a first machine learning algorithm that is trained to identify odors on the basis of the first signal that comprise the odors from alcohol, cocaine, amphetamines, cigarette smoke, cannabis, tetrahydrocannabinol, morphine, methadone, ammonia, acetone, or a combination of these substances. The odors are assigned to the identity of a vehicle occupant in step V4 on the basis of the position of the vehicle occupant F in the vehicle interior. A second signal is generated in the case of a positive assignment in step V5. The second signal is sent to at least one vehicle unit in step V6.

The method is carried out by the processing unit 10 or the system 20.

### REFERENCE SYMBOLS

F vehicle occupant

10 processing unit

11 first interface

12 second interface

13 third interface

20 system

21 first sensor

22 second sensor

ECU vehicle control system

V1-V6 steps of the method

1. A processing unit for a vehicle interior for detecting and reaction to odors of a vehicle occupant, comprising:
a first interface to a sensor that is configured to detect odor molecules in the exhalations and/or body odor of the vehicle occupant and convert the odor molecules to a first signal to obtain the first signal, wherein the first signal results from light interactions or comprises frequencies and/or amplitudes of oscillations;
a second interface to a second sensor that is configured to identify at least one vehicle occupant in order to obtain at least one identity of the at least one vehicle occupant;
wherein the processing unit carries out commands with which the processing unit:
assigns odors indicating drugs and/or illnesses to the at least one identity of the at least one vehicle occupant on a basis of a position of the at least one vehicle occupant in the vehicle interior, and
generates a second signal in response to a positive assignment, wherein the commands comprise a first machine learning algorithm that is trained to identify at least one of alcohol, cocaine, amphetamines, cigarette smoke, cannabis, tetrahydrocannabinol, morphine, methadone, ammonia, acetone, or a combination of these substances in the odors of the at least one vehicle occupant on a basis of the first signal, and
a third interface to at least one vehicle unit configured to provide the second signal to the at least one vehicle occupant and/or a vehicle control system,
wherein the first machine learning algorithm is trained in semantic image segmentation to identify the at least one vehicle occupant, or the commands comprise a second machine learning algorithm that is trained in semantic image segmentation to identify the at least one vehicle occupant.

2. The processing unit according to claim 1, wherein the first machine learning algorithm simulates a mammalian olfactory system and/or the processing unit forms a neuromorphic circuit.

3. The processing unit according to claim 1, wherein the processing unit is integrated in a vehicle or a vehicle electrical system.

4. A system comprising:
at least one first sensor configured to detect odor molecules in the exhalations and/or body odor of the at least one vehicle occupants, and convert the odor molecules into first signals;
at least one second sensor configured to identify the at least one vehicle occupants; and
the processing unit according to claim 1, which is connected for signal transfer to the at least one first sensor and the at least one second sensor via the first interface and the second interface, respectively, wherein
the system is configured to send a second signal from the processing unit to an optical, acoustic, and/or tactile information unit or vehicle control unit via the third interface.

5. The system according to claim 4, wherein the first sensor is a vehicle lidar sensor configured to detect odor molecules on a basis of diffused light returning from the vehicle interior.

6. The system according to claim 5, wherein the vehicle lidar sensor is configured to emit numerous light pulses of different wavelengths, and detect the odor molecules form the returning light for each of the wavelengths.

7. The system according to claim 4, wherein the vehicle lidar sensor comprises Q-switching.

8. The system according to claim 4, wherein the second sensor is a 2D or 3D camera sensor, radar sensor, or lidar sensor, and the system is configured to identify the at least one vehicle occupant through facial recognition.

9. The system according to claim 4, comprising a particular sensor that unifies functionalities of the at least one first sensor and the at least one second sensor, wherein the particular sensor is a time-of-flight sensor, in which the first signals are a result of light returning to the particular sensor form the vehicle interior, and the at least one vehicle occupant is identified on a basis of the time-of-flight for light pulses from the particular sensor to a body surface on the vehicle occupant.

10. The system according to claim 8, wherein the at least one second sensor detects individual appendages, and the system is configured to identify the at least one vehicle occupant on a basis of the detected appendages.

11. The system according to claim 4, wherein the at least one first sensor is located on a vehicle steering wheel or in the dashboard, and the at least one second sensor is located on the vehicle steering wheel, in the dashboard, in the windshield, or in the roof of the vehicle, when used in the vehicle interior.

12. The system according to claim 4, wherein the at least one first sensor is a vehicle lidar sensor configured to detect odor molecules on a basis of light returning from the vehicle interior, and the at lest one second sensor is a CCD or CMOS sensor for a spectroscopic analysis of the returning light.

13. The system according to claim 4, wherein the second signal is a control signal for an immobilizer in the vehicle, and the third interface provides the second signal to the immobilizer, or wherein the second signal is a control signal, and triggers a fail-safe or fail-operational state for the vehicle.

14. The system according to claim 4, comprising a memory and/or communication means for storing identified odors from drugs and/or illnesses and/or sending information regarding the identified odors to a business to which the vehicle belongs, or to a government agency.

15. A computer-implemented method for detecting and reacting to odors of at least one vehicle occupant, comprising:
obtaining, by a processing unit, first signals that describe odor molecules;
obtaining, by the processing unit, at least one identity identities of the at least one vehicle occupant;
identifying, by the processing unit, odors indicating drugs and/or illnesses on a basis of the first signals, using a first machine learning algorithm that is trained to identify odors comprising the odors of at least one of alcohol, cocaine, amphetamines, cigarette smoke, cannabis, tetrahydrocannabinol, morphine, methadone, ammonia, acetone, or combinations of these substances on the basis of the first signals;
assigning, by the processing unit, the odors to the at least one identity of the at least one vehicle occupant on a basis of a position of the at least one vehicle occupant in the vehicle interior;
generating, by the processing unit, a second signal in in response to a positive assignment; and
providing, by the processing unit, the second signal to at least one vehicle unit.

16. The system according to claim 9, wherein the at least one first sensor is located on a vehicle steering wheel or in the dashboard, and the particular sensor is located on the vehicle steering wheel, in the dashboard, in the windshield, or in the roof of the vehicle, when used in the vehicle interior.
